(19) United States
(12) Patent Application Publication
(10) Pub. No.: US 2003/0004523 A1
(43) Pub. Date: Jan. 2, 2003

(54) Multi-needle holding device

(75) Inventors: Peter T.K. Chan, Quebec (CA); Philip S. Li, Douglaston, NY (US); Marc Goldstein, New York, NY (US)

Correspondence Address: HOFFMANN & BARON, LLP, 6900 JERICHO TURNPIKE, SYOSSET, NY, 11791, US

(73) Assignee: Cornell Research Foundation, Inc.

(21) Appl. No.: 10/187,699

(22) Filed: Jul. 2, 2002

Related U.S. Application Data
(60) Provisional application No. 60/302,403, filed on Jul. 2, 2001.

(51) Int. Cl. A61B 17/04

(52) U.S. Cl. 606/148

(57) ABSTRACT

A needle holding device includes a handle, a pivot point and two opposing jaws opposite the handle and pivotable about the pivot point. The jaws have inner opposing surfaces and at least one inner opposing surface has at least one needle groove formed therein for receiving and gripping a needle. At least one of the opposing surfaces has a knurled or rugged platform for securely gripping at least one needle. The needle holding device may have a plurality of substantially parallel or non-parallel needle grooves formed therein for simultaneously receiving and gripping a plurality of needles. Additionally, the plurality of non-parallel needle grooves preferably includes an innermost needle groove disposed nearest to the pivot point and an outermost needle groove disposed furthest from the pivot point, wherein the plurality of needle grooves successively increase in depth from the outermost needle groove to the innermost needle groove so that a plurality of needles can be simultaneously held regardless of needle size or position within the holder.

CROSS-REFERENCE TO RELATED APPLICATIONS

This application claims the benefit of U.S. Provisional Application No. 60/302,403, filed on Jul. 2, 2001.

### FIELD OF THE INVENTION

The present invention relates generally to surgical needle holders and more particularly to an improved needle holder providing a more stable platform on which to mount a needle during laparoscopy or microsurgery. The device further allows for simultaneous placement of multiple needles thereby reducing operating time and providing for more precise suture alignment.

### BACKGROUND OF THE INVENTION

With the tremendous increase in surgeons' experiences on minimal invasive surgery performed laparoscopically and microsurgically, the safety and outcome of these newest forms of surgical treatment options have improved dramatically in recent years. At the same time, there has been an increase in the demand both from patients in receiving surgical care laparoscopically and microsurgically when indicated, and from surgeons in acquiring such surgical techniques.

In recent years, laparscopic surgery has shifted from a resection-oriented phase to a reconstruction-oriented one, requiring anastomosis to be made to a variety of structures with conventional suture materials. The action of needle placement laparscopically is a technical challenge. The insertion and removal of the laparscopic instrument to and from the inflated peritoneum for mounting each needle is tedious and time consuming.

Furthermore, it has been clearly established in the current medical literature that micro-surgical reconstruction of the male reproductive tract with vasectomy or vasoepididymostomy in men with infertility due to obstruction is a more cost-effective management than the use of assisted reproductive technology. Currently, 500,000 men per year in the United States choose vasectomy as a means of permanent contraception. In part because of a high divorce rate, surveys estimate that 10,000 to 30,000 men per year seek vasectomy reversal.

Berger (J. Urol. 1998, 159: 1951) described a “triangulation” technique which was evaluated as having superior results (J. Urol. 1999, 161 (Suppl): 93; J. Urol. 1999, 161 (Suppl): 350). This technique was later modified by Marmar (J. Urol. 2000, 163: 483) into a “two-suture” anastomosis this latter modification requires precise and simultaneous placement of two fine micro-needles and is technically difficult to perform even for experienced microsurgeons.

Accordingly, it would be desirable to provide a needle holder for precision placement and secure holding of one or more needles, thereby significantly reducing the operating time spent on remounting needles and, more importantly, making multi-suture placement more precise and easier.

### SUMMARY OF THE INVENTION

The present invention is a needle holding device including a handle, a pivot point and two opposing jaws opposite the handle and pivotable about the pivot point. The jaws have inner opposing surfaces and at least one inner opposing surfaces has at least one needle groove formed therein for receiving and gripping a needle.

In a preferred embodiment, the needle holding device is in the form of a scissors-type surgical clamp and at least one of the opposing surfaces has a knurled or rugged platform for securely gripping at least one needle. The needle holding device further preferably includes a biasing element, such as a spring, for urging the inner opposing surfaces together.

The needle holding device may take the form of a parallel needle holder, wherein at least one of the opposing surfaces has a plurality of substantially parallel needle grooves formed therein for simultaneously receiving and gripping a plurality of needles. The plurality of parallel needle grooves may be spaced equidistant apart so that a variety of multiple needle spacings can be achieved. Additionally, the plurality of parallel needle grooves preferably includes an innermost needle groove disposed nearest to the pivot point and an outermost needle groove disposed furthest from the pivot point, wherein the plurality of needle grooves successively increase in depth from the outermost needle groove to the innermost needle groove so that a plurality of needles can be simultaneously held regardless of needle size or position within the holder.

Alternatively, the needle holding device may take the form of an angled needle holder, wherein at least one of the opposing surface has a plurality of non-parallel needle grooves formed therein for simultaneously receiving and gripping a plurality of needles. The non-parallel needle grooves are preferably formed at known angles with respect to each other. Preferably, the non-parallel needle grooves are at a minimum of about 1° with respect to each other and a maximum of about 90° with respect to each other. Again, the plurality of non-parallel needle grooves preferably includes an innermost needle groove disposed nearest to the pivot point and an outermost needle groove disposed furthest from the pivot point, wherein the plurality of needle grooves successively increase in depth from the outermost needle groove to the innermost needle groove so that a plurality of needles can be simultaneously held regardless of needle size or position within the holder.

A preferred embodiment of the improved needle holder, as well as other features and advantages of this invention, will be apparent from the following detailed description, which is to be read in conjunction with the accompanying drawings.

### BRIEF DESCRIPTION OF THE DRAWINGS

FIG. 1 is a perspective view of the multi-needle holding device formed in accordance with the present invention.

FIG. 2 is a detailed perspective view of the jaws of the needle holding device shown in FIG. 1.

FIG. 3 is a plan view of one of the jaws of the needle holder shown in FIGS. 1 and 2.

FIG. 4 is a perspective view of the jaws of an alternative embodiment of the needle holder formed in accordance with the present invention.

FIG. 5 is a plan view of one of the jaws of the needle holder shown in FIG. 4.

FIGS. 6a-6c are side views of the jaws of the needle holder shown in FIGS. 1-3 showing placement of different sized needles.

FIG. 7 illustrates use of the needle holder shown in FIGS. 1-3 where a small opening in a tubular structure is desired.

FIG. 8 illustrates use of the needle holder shown in FIGS. 1-3 where a large opening in a tubular structure is desired.

FIG. 9 illustrates use of the needle holder shown in FIGS. 4-5.

### DETAILED DESCRIPTION OF THE PREFERRED EMBODIMENT

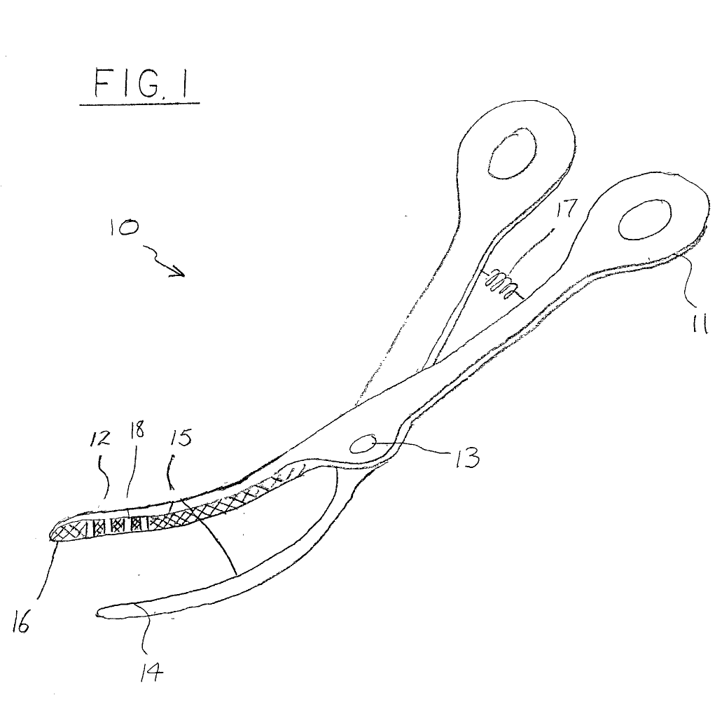

Referring first to FIG. 1, a parallel multi-needle holding device 10 according to the present invention is shown. The device 10 is generally similar to a conventional scissors-type surgical clamp comprising two opposing jaws 12 and 14 pivotable about a pivot point 13, and a handle 11 for opening and closing the jaws. At least one, and preferably both, of the inner opposing surfaces 15 of the opposing jaws 12 and 14 are preferably formed having a rugged or knurled platform 16 for securely holding a single needle or multiple needles. The rugged platform portion 16 provides for stronger grip and strength in needle placement than can be provided by traditional needle holders. Additionally, the device 10 further preferably includes a biasing element 17 for urging the inner opposing surfaces 15 together. The biasing element 17 may be a spring located near the handle 11 for urging the opposing jaws 12 and 14 into a normally closed position, wherein the inner opposing surfaces 15 may securely grip one or more needles.

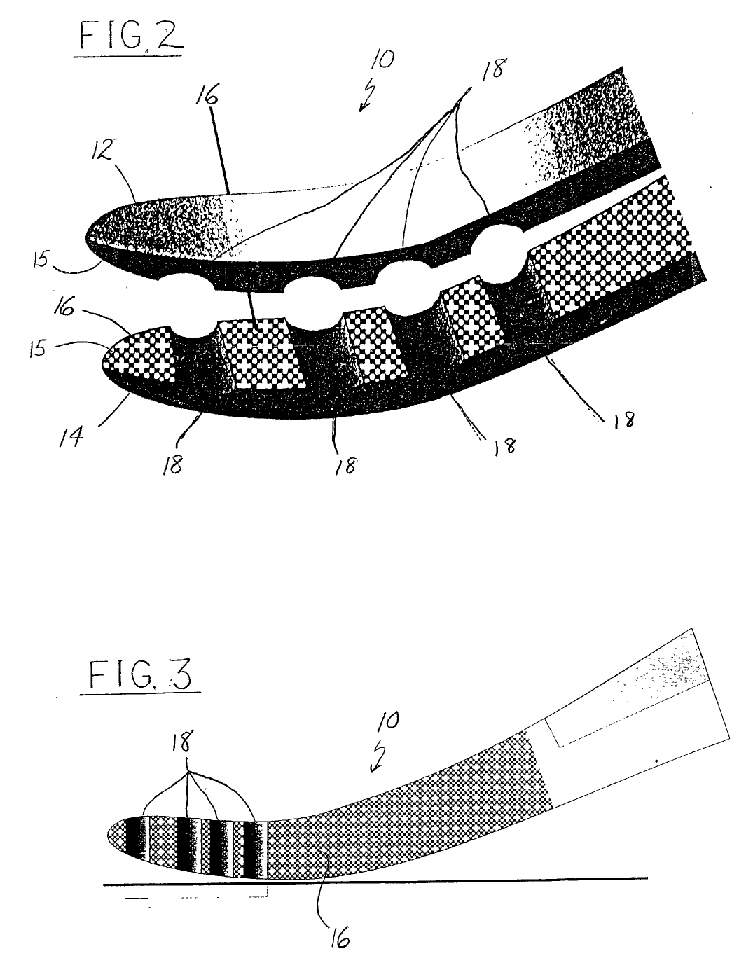

Referring additionally to FIGS. 2 and 3, in the preferred embodiment, at least one, and preferably both, of the inner opposing surfaces 15 of both opposing jaws 12 and 14 are formed having at least one needle groove 18 for receiving and gripping at least one needle. Thus, a single needle or multiple needles can be mounted directly on the rugged platform portion 16 of the jaws 12 and 14 or can be mounted in one of the grooves 18 or any combination thereof. The combination of the rugged platform 16 and the needle groove 18 provides flexibility of use for the surgeon. The needle holding device 10 can be used as a regular needle holder for a single needle or, where multiple grooves 18 are provided, can be used with multiple needles. Thus, the additional needle groove 18 provides improved grip, as well as extra precision during needle placement.

With traditional micro-needle holders, because of insecure needle grip, the micro-needles will often “spring off” from the needle holder. It is extremely time consuming, if not impossible, to look for the lost needle. The needle holder of the present invention provides for secure gripping and, therefore, the chance of accidental “springing off” is reduced.

In the embodiment shown in FIGS. 1-3, the needle holding device 10 is formed with a plurality of needle grooves 18 which are substantially parallel to one another for allowing perfect parallel placement of multiple needles. In addition, the parallel grooves will secure the direction of the needles and prevent unintentional disturbance of the entrance angle during needle placement. Perfect parallel alignment of multiple needles will also provide equal anastomotic tension for tying sutures. As will be discussed in further detail below, the depth and spacing of the grooves 18 is predetermined to allow for a variety of needle sizes and spacings.

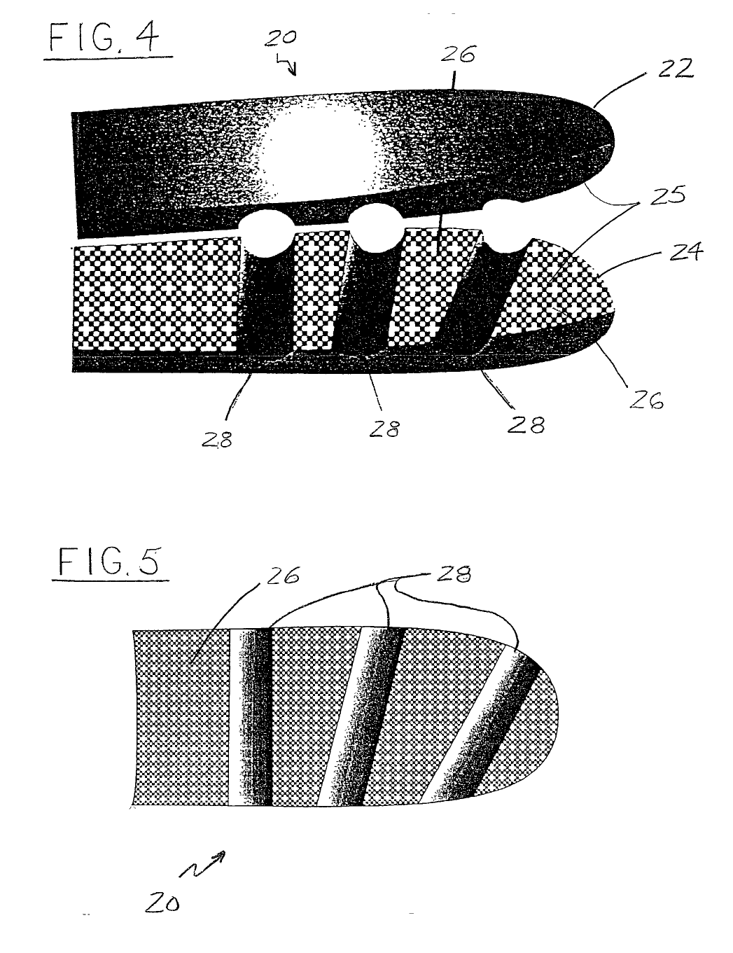

Referring now to FIGS. 4 and 5, an alternative embodiment of a multi-needle holder 20 of the present invention is shown. The multi-needle holder 20 shown in FIGS. 4 and 5 is in the form of an angled needle holder. Again, the angled needle holder 20 is similar to a conventional surgical clamp having opposing upper and lower jaws 22 and 24 and a handle (not shown in FIGS. 4 and 5) for opening and closing the jaws. Similar to the needle holding device 10 described above, at least one, and preferably both, of the opposing inner surfaces 25 of the jaws 22 and 24 of the angled needle holder 20 are formed with a rugged or knurled platform 26 and at least one needle groove 28. However, unlike the parallel needle holding device 10, the angled needle holder 20 includes a plurality of nonparallel needle grooves 28 formed in the jaws 22 and 24 at predetermined angles with respect to each other. The minimum angle between the nonparallel grooves 28 is about 1 degree, while the maximum angle between the nonparallel grooves is about almost 90 degrees. This allows for tangential needle entrance to the tissue plane during multiple needle placement.

Like the parallel needle holding device 10, the angled needle holding device 20 can be used in a conventional manner by simply positioning and gripping one or more needles between the upper and lower jaws 22 and 24. The rugged platform 26, however, provides superior grip with the present invention. Alternatively, one or more needles can be positioned in the needle grooves 28 to provide extra precise positioning of the needle(s).

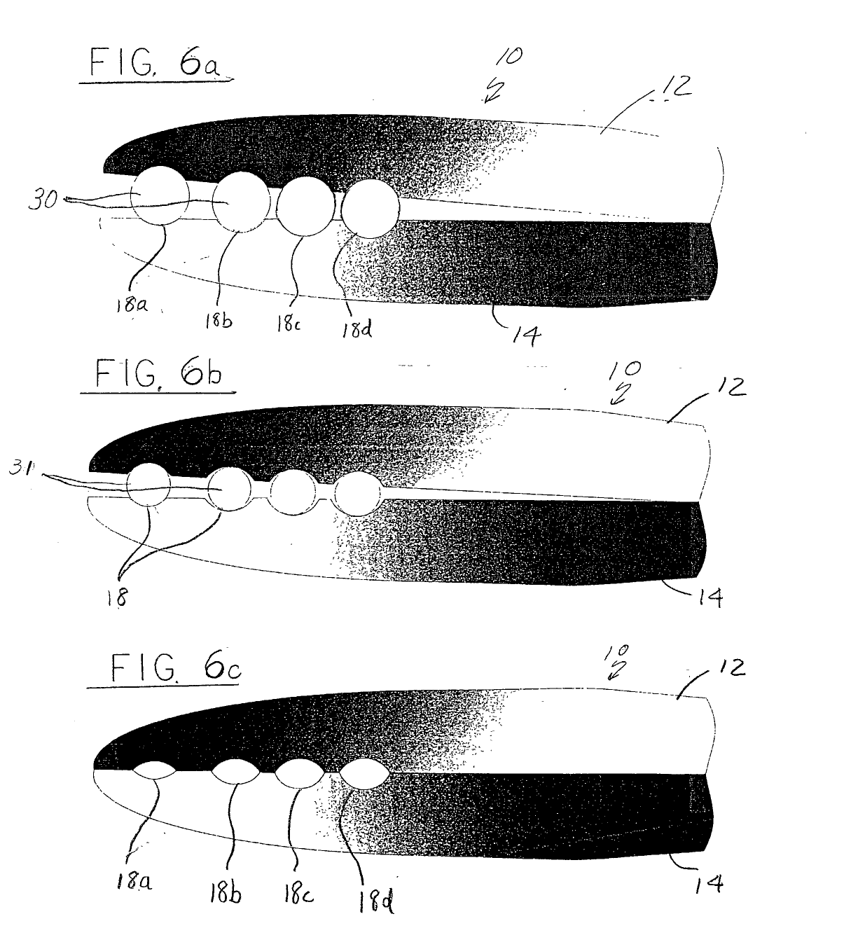

Referring now to FIGS. 6a-c, the needle grooves 18 and 28 for both the parallel needle holder 10 and the angled needle holder 20 are specially designed such that all of the grooves provide similarly secure grip to the needle regardless of how many needles are being held at one time. FIG. 6a shows a parallel needle holder 10 holding four 9-0 (100 μm) size needles 30, one in each of four needle grooves 18. FIG. 6b shows a parallel needle holder 10 holding four 10-0 (70 μm) needles 31, one in each of four needle grooves 18. (For the angled needle holder 20, the needle grooves 28 would typically be designed for needles of sizes 2-0 to 4-0, each being held securely within the device regardless of size.) As can be seen in these Figures, each of the needles is securely held regardless of its size or which groove it is positioned. This can be accomplished by decreasing the depth of each successive groove from the innermost groove 18d (i.e., closest to the device pivot point 13) to the outermost groove 18a as illustrated in FIG. 6c showing the device 10 fully closed.

Furthermore, particularly for the parallel needle holder 10, the grooves 18a-d are spaced at predetermined distances so that a multitude of needle spacing configurations can be achieved. For example, if the spacing is equidistant, placing needles in grooves 18a and 18b will achieve a spacing of 1.0 unit. By placing needles in grooves 18a and 18c, a spacing of 2.0 units can be achieved and so on.

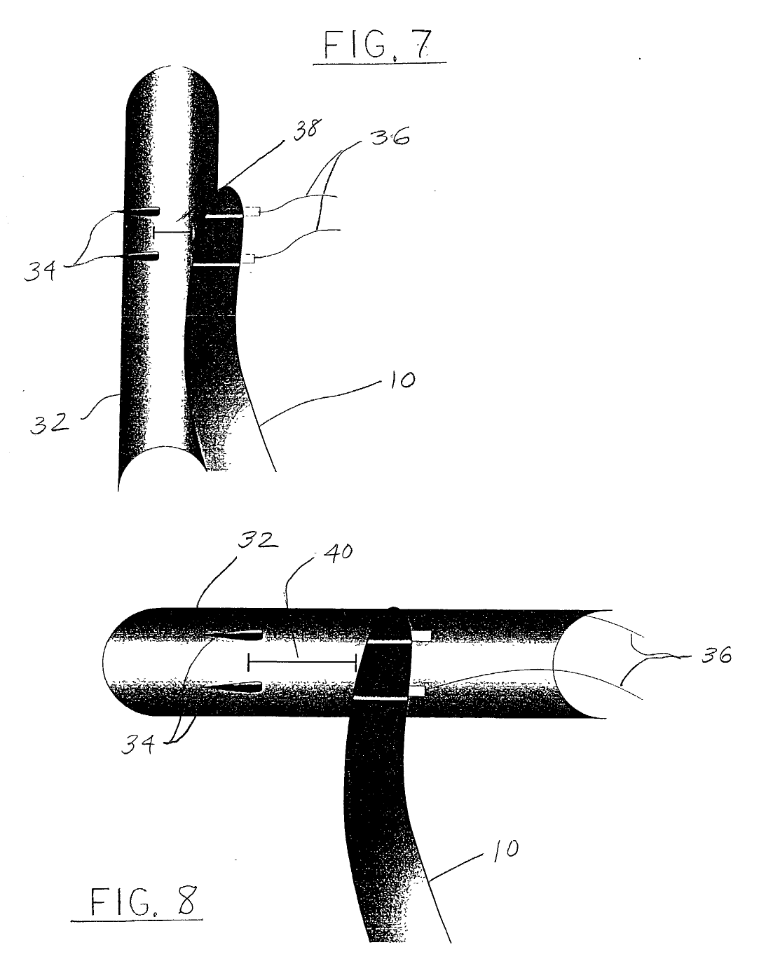

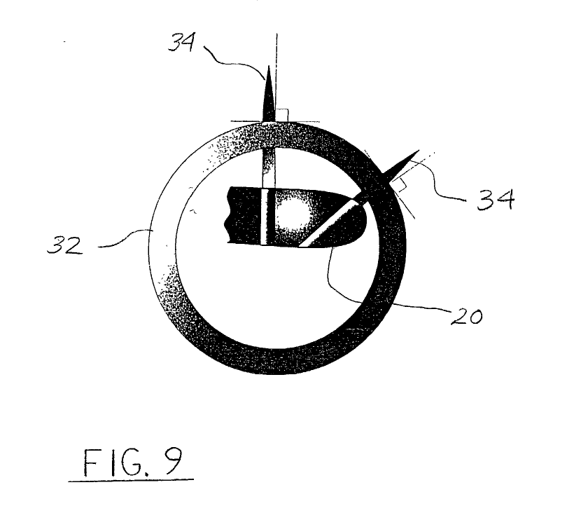

FIGS. 7 and 8 show use of a parallel needle holding device 10 formed in accordance with the present invention, while FIG. 9 shows use of an angled needle holding device 20 according to the present invention. As shown in FIGS. 7-9, both devices 10 and 20 are ideal for double-suture 36 placement. Additionally, both devices provide more secure grip to the distal needle than traditional needle holders. With the parallel needle holding device 10, as shown in FIGS. 7 and 8, two micro-needles 34 can be placed simultaneously in parallel fashion. As shown in FIG. 7, when a small opening 38 in the tubular structure 32 is desired, the parallel needle holding device 10 is positioned so that the needles 34 are placed on the epididymal tubule perpendicularly, as done in a conventional double-suture vasoepididymostomy. However, as shown in FIG. 8, the parallel needle holding device 10 of the present invention allows for two micro-needles 34 to be precisely placed simultaneously along the epididymal tubule in a longitudinal fashion. This allows a larger opening for anastomosis, thereby enhancing the post-operative patency rate.

FIG. 9 shows use of the angled needle holding device 20 according to the present invention wherein the pre-designed angles between the grooves 28 allow for tangential needle entrance to the tissue plane. Simultaneous placement of two needles 34 in this manner will reduce the number of maneuvers, making the surgery easier especially for surgeons who perform such surgeries only occasionally. For experienced surgeons, it will significantly reduce the operating time while providing a more precise suture alignment.

The needle holders of the present invention solve the tedious and time consuming problem of preciously mounting individual needles. The invention also allows placement of more than one needle, with a more secure grip and in a pre-set direction. The needle holders of the present invention will significantly reduce the operating time spent on remounting needles and, more importantly, will make multi-suture placement more precise and easier. The needle holders of the present invention have tremendous application in the fields of laproscopic, micro, vascular, ophthalmologic, neurological and plastic surgery where absolute precision is required.

Although the illustrative embodiments of the present invention have been described herein with reference to the accompanying drawings, it is to be understood that the invention is not limited to those precise embodiments and various other changes may be effected therein by one skilled in the art without departing from the scope or spirit of the invention.

What is claimed is:

1. A needle holding device comprising:
a handle;
a pivot point; and
two opposing jaws opposite said handle and pivotable about said pivot point, said jaws having inner opposing surfaces, wherein at least one inner opposing surface includes at least one needle groove formed therein for receiving and gripping a needle.

2. A needle holding device as defined in claim 1, wherein at least one opposing surface is knurled.

3. A needle holding device as defined in claim 1, wherein said at least one opposing surface has a plurality of substantially parallel needle grooves formed therein for simultaneously receiving and gripping a plurality of needles.

4. A needle holding device as defined in claim 3, wherein said plurality of parallel needle grooves are spaced equidistant apart.

5. A needle holding device as defined in claim 3, wherein said plurality of parallel needle grooves comprises an innermost needle groove disposed nearest to said pivot point and an outermost needle groove disposed furthest from said pivot point, said plurality of needle grooves successively increasing in depth from said outermost needle groove to said innermost needle groove.

6. A needle holding device as defined in claim 1, wherein said at least one opposing surface has a plurality of non-parallel needle grooves formed therein for simultaneously receiving and gripping a plurality of needles.

7. A needle holding device as defined in claim 6, wherein said plurality of non-parallel needle grooves comprises an innermost needle groove disposed nearest to said pivot point and an outermost needle groove disposed furthest from said pivot point, said plurality of needle grooves successively increasing in depth from said outermost needle groove to said innermost needle groove.

8. A needle holding device as defined in claim 6, wherein said plurality of non-parallel needle grooves are formed at predetermined angles with respect to each other.

9. A needle holding device as defined in claim 8, wherein the minimum angle between any two of said nonparallel needle grooves is about 1 degree.

10. A needle holding device as defined in claim 8, wherein the maximum angle between any two of said nonparallel needle grooves is about 90 degrees.

11. A needle holding device as defined in claim 1, wherein said needle holding device is in the form of a scissors-type surgical clamp.

12. A needle holding device as defined in claim 1, further comprising a biasing element for urging said inner opposing surfaces together.

13. A needle holding device as defined in claim 11, wherein said biasing element is a spring.

14. A jaw for a needle holding device comprising an inner surface and at least one needle groove formed in said inner surface for receiving and gripping a needle.

15. A jaw for a needle holding device as defined in claim 14, wherein said inner surface is knurled.

16. A jaw for a needle holding device as defined in claim 14, wherein said inner surface has a plurality of substantially parallel needle grooves formed therein for simultaneously receiving and gripping a plurality of needles.

17. A jaw for a needle holding device as defined in claim 16, wherein said plurality of parallel needle grooves are spaced equidistant apart.

18. A jaw for a needle holding device as defined in claim 16, wherein said plurality of parallel needle grooves comprises an innermost needle groove and an outermost needle groove, said plurality of needle grooves successively increasing in depth from said outermost needle groove to said innermost needle groove.

19. A jaw for a needle holding device as defined in claim 14, wherein said inner surface has a plurality of non-parallel needle grooves formed therein for simultaneously receiving and gripping a plurality of needles.

20. A jaw for a needle holding device as defined in claim 19, wherein said plurality of non-parallel needle grooves comprises an innermost needle groove and an outermost needle groove, said plurality of needle grooves successively increasing in depth from said outermost needle groove to said innermost needle groove.
